(19) United States
(12) Patent Application Publication
(10) Pub. No.: US 2011/0065776 A1
(43) Pub. Date: Mar. 17, 2011

(54) Method for Treating Hepatitis C Infection

(76) Inventors: Keng-Hsin Lan, Taipei City (TW); Keng-Li Lan, Taipei City (TW)

(21) Appl. No.: 12/884,909

(22) Filed: Sep. 17, 2010

Related U.S. Application Data
(60) Provisional application No. 61/243,380, filed on Sep. 17, 2009.

(51) Int. Cl. A61K 31/713; A61P 31/14; C12Q 1/48

(52) U.S. Cl. 514/44 A; 435/15

(57) ABSTRACT

The present invention relates to a method for treating hepatitis C virus (HCV) infection, comprising administrating a subject in need thereof with a therapeutically effective amount of an inhibitor against a serine/threonine kinase (AKT) and an activator thereof. A method for screening a candidate agent for treating hepatitis C infection determined by the presence of an inhibition of AKT function is also provided.

### CROSS-REFERENCE TO RELATED APPLICATION

This application claims priority benefit of U.S. Provisional Application Ser. No. 61/243,380, filed Sep. 17, 2009, which application is incorporated herein by reference in its entirety.

### FIELD OF THE INVENTION

The present invention is related to a new approach for the treatment of hepatitis C infection. Particularly, the present invention is related to a method for treating patients suffering from hepatitis C infection, and a method for screening an active agent for treating hepatitis C infection.

### BACKGROUND OF THE INVENTION

Hepatitis C virus (HCV) is recognized as a major causative agent of chronic hepatitis, cirrhosis, and hepatocellular carcinoma (Schutte et al., Hepatocellular carcinoma—epidemiological trends and risk factors. Dig Dis 27(2):80-92, 2009). Based on genetic differences between HCV isolates, there are six genotypes with several subtypes. Genotype is clinically important in determining potential response to interferon-based therapy and the required duration of such therapy. The most prevalent ones are genotype 1 and 2. However, the genotype 1 HCV as compared with the genotype 2 HCV infection is characterized by less sustained virological response (SVR) with treatment of interferon/ribavirin and more frequent incidence of hepatocellular carcinoma development in HCV carriers. Therefore, infection of HCV genotype 1 represents greater challenge for decreasing viral load using standard treatment with regimen of interferon in combination of protease inhibitors, such as ribavirin (Kronenberger and Zeuzem. Current and future treatment options for HCV, Ann Hepatol 8(2):103-12, 2009). However, interferon and ribavirin are not specific antiviral agents designed for HCV, and they have experienced drug resistance and therapeutic failure in a significant portion of patients.

Proteins made by HCV include structural proteins E1 and E2, and nonstructural proteins NS2, NS3, NS4, NS4A, NS4B, NS5, NS5A, and NS5B. HCV NS5B is a putative serine phosphoprotein, which is a viral RNA-dependent RNA polymerase (RdRP) required for replication of HCV RNA genome. On the other hand, HCV NS3 is a multifunctional protein containing an amino-terminal serine protease and a carboxy-terminal helicase/nucleoside triphosphatase domain. The NS3 serine protease is essential for post-translational processing of the NS3-NS5 region of the HCV polyprotein to produce components of the viral RNA replication complex. The helicase plays an important role in viral replication by unwinding the viral RNA. Given the critical roles of NS5B and NS3 in HCV replication, numerous compounds targeting the functions of these two proteins are currently in clinical trials (for example, De Francesco and Carfi. Advances in the development of new therapeutic agents targeting the NS3-4A serine protease or the NS5B RNA-dependent RNA polymerase of the hepatitis C virus. Adv. Drug Deliv. Rev. 59(12):1242-62, 2007; and Kwong et al. Recent progress in the development of selected hepatitis C virus NS3.4A protease and NS5B polymerase inhibitors; Current opinion in pharmacology 8(5):522-31, 2008).

There is still a great need for a novel and more effective approach for treatment of HCV infections is still needed.

### BRIEF SUMMARY OF THE INVENTION

The present invention provides a new approach for treatment of Hepatitis C virus (HCV) infection. It is unexpectedly discovered in the present invention that an inhibitor of the activation of a serine/threonine kinase (AKT) could block the replication of HCV through suppression in HCV NS3 and NS5B function; therefore, an inhibitor targeting AKT1 and its related activating pathways could serve as an agent for treatment of HCV infection.

In one aspect, the invention provides a method for treating hepatitis C virus (HCV) infection. The method comprises administrating a subject in need thereof with an inhibitor of the activation of a serine/threonine kinase (AKT) and its activators, at an amount effective for blocking the function of NS3 or NS5B of HCV.

In another aspect, the invention provides a method for treating hepatitis C virus (HCV) infection with minimal side effects, comprising administrating a subject in need thereof with an agent that inhibits the activation of AKT1 but not AKT2, at an amount effective for blocking the function of NS3 or NS5B of HCV.

In one example of the invention, the side effects on insulin function are minimized.

In further aspect, the invention provides a method for screening a candidate agent for treating HCV infection, comprising:

determining the function of a candidate agent to inhibit the activity of a serine/threonine kinase (AKT) and detecting the presence or absence of an inhibition of the function of AKT; wherein the more function to inhibit the activation of AKT indicates the more activity in treating HCV infection.

In yet aspect, the invention provides a method for screening a candidate agent for treating HCV infection with minimal side effects, comprising: determining the function of a candidate agent to inhibit specifically the activity of AKT1 and AKT2; and detecting the presence or absence of an inhibition of the function of AKT1 and AKT2, respectively; wherein the more function to inhibit the activation of AKT1 but not AKT2 indicates the more activity in treating HCV infection with minimal side effects.

### BRIEF DESCRIPTION OF THE SEVERAL VIEWS OF THE DRAWINGS

For the purpose of illustrating the invention, there are shown in the drawings embodiments. It should be understood, however, that the invention is not limited to the preferred embodiments shown.

In the drawings:

FIG. 1A provides a comparison of the amino-acid sequence of the AKT-phosphorylation motif of HCV genotype 1b NS5B and those of known AKT substrates;

FIG. 1B provides a sequence alignment of the sequences of putative AKT-phosphorylation motifs of the potential phosphorylation residues shown in various HCV NS5B, wherein the numbers on the top indicate the positions of the potential phosphorylation residues, respectively;

FIG. 2A provides the association between HCV NS5B and AKT, wherein Flag-tagged HCV NS5B, HCV NS5B mutant or vector and HA-tagged Myr-AKT transfecting into 293T cells were immunoprecipitated with anti-Flag-M2 antibody and immunoblotted (IB) wutg anti-HA (3F10) antibody; and Lanes 4, 5, and 6 represented 5% of the cell lysates used for coimmunoprecipitation;

FIG. 2B provides the association between HCV NS5B and AKT/HA, wherein the 293T cell lysates were immunoprecipitated with anti-AKT or anti-HA (3F10) antibody, and detected with FLAG-specific antibody, wherein Lanes 7, 8, and 9 represented 5% of the cell lysates used for coimmunoprecipitation;

FIG. 2C provides the association between HCV NS5B-1b/NS5B-2b and AKT wherein Flag-tagged Ns5B-1b, NS5B-2B or FLAG vector were co-transfected with HA-tagged AKT into Huh7 cells, immunoprecipitated with anti-FLAG and immunoblotted with anti-HA antibodies, showing that the AKT was specifically immunoprecipitated by NS5B-1b and NS5B-2b but not the vector control, and the equal expression level of AKT representing 5% of the cell lysates used for coimmunoprecipitation was shown in lower panel;

FIG. 2D provides the results of the immunoprecipitation with anti-HA (upper panel) or anti-AKT (middle panel) antibodies of the Huh7 cell lysates co-transfected with FLAG-NS5B-1b, FLAG-NS5B-2b or FLAG vector, and the equal expression level of NS5B-1b and NS5B-2b representing 5% of the cell lysates (lower panel);

FIG. 2E provides the results of NS5B-1b, NS5B-2b and vector immunoprecipitated with anti-FLAG and immunoblotted with anti-HA antibodies;

FIG. 2F shows the results of NS5B-1b, NS5B-2b and vector immunoprecipitated with AKT, Myr-AKT and DN-AKT and immunoblotted with anti-PLAG;

FIG. 3 provides the in vitro interaction of HCV NS5B with AKT, wherein the same membrane was re-probed with anti-GST antibody, indicating that the amount of GST or GST fusion protein was pulled down (middle panel), equal to that of 5% of the cell lysates (lower panel);

FIG. 4 provides the interaction of AKT with C-terminus of HCV NS5B, wherein the specific co-immunoprecipitation of AKT-HA (upper panel) and phosphorylated AKT (second panel) were only shown by full-length (aa 1-591) and C-terminal of NS5B (aa 372-591) but not FLAG vector or N-terminal NS5B (1-371);

FIG. 5A provides an in vitro phosphorylation of NS5B at serine 506 by AKT;

FIG. 5B provides a phosphorylation of NS5B at serine 513 by AKT; wherein the arrows indicated the positions of NS5B phosphorylated by AKT and autophosphorylated AKT; the phosphorylation of wild-type NS5B, NS5B (S506A), and NS5B (S513A) by AKT was clearly observed (Lane 3, 5 and 7) while NS5B carrying the double mutations at the potential AKT phosphorylation consensus motif S506,513AA) entirely prevented AKT phosphorylation (Lane 9), and the Sf9 cell lysates were used as negative control (Lane 1 and 2);

FIG. 6 provides the in vivo results of the phosphorylation of NS5B; wherein the phosphorylated NS5B was specifically immunoprecipitated from 293T/FLAG-NS5B cell lysates (upper panel), the membrane was reprobed with anti-FLAG M2 antibodies to show NS5B expression in 293T/FLAG-NS5B cells (middle panel), and the equal expression of phosphorylated AKT was shown in immunoblot of input using anti-phospho-AKT (S473) antibodies (lower panel);

FIG. 7A provides a comparison of the amino-acid sequence of the AKT-phosphorylation motif of HCV genotype 1b NS3 and those of known AKT substrates;

FIG. 7B provides a sequence alignment of putative AKT-phosphorylation motifs sequences among several genotypes of HCV NS3, wherein the numbers on the top indicate the positions of the potential phosphorylation residues, respectively;

FIG. 8A provides the in vivo interaction of HCV NS3 M-terminal region with AKT, wherein flag-tagged HCV NS3 or vector and HA-tagged AKT were co-transfected into 293T cells, the cells were immunoprecipitated with anti-FLAG-M2 antibody, and AKT was immunoblotted with anti-HA (3F10) antibody;

FIG. 8B provides the results for determining the region required for NS3 interaction with AKT, wherein the 293T cells lysates after transfection with the flag-tagged constructs encoding various regions of NS3 with the full length (1-631), a first fragment (1-191) and a second fragment (192-631) respectively, were immunoprecipitated with anti-FLAG-M2 antibody, and AKT was immunoblotted with anti-HA (3F10) antibody;

FIG. 9 shows that NS3 was phosphorylated at serine residue within the RXRXXS/T AKT phosphorylation motif,

FIG. 10 shows that Serine 122 in the AKT-phosphorylation motif RXRXXS/T of NS3 was phosphorylated by active AKT, wherein the arrows indicated the positions of NS3 phosphorylated by AKT and autophosphorylated AKT, and the phosphorylation of wild-type NS3 by AKT was clearly observed in the presence of active AKT (Lane 3) while the mutation of NS3 at Serine 122 to Alanine in the AKT phosphorylation consensus motif entirely prevented AKT phosphorylation (Lane 5 and 6), and Sf9 cell lysates were used as negative control (Lane 1 and 2); and

FIG. 11 shows the suppression of NS3 activity by elimination of AKT-phosphorylation motif; wherein the AKT-phosphorylation motif eliminated S122A mutant demonstrated suppressed SEAP activity as compared with wild type, indicating decreased NS3 activity in the absence of AKT phosphorylation of S122; and

FIG. 12 shows the regulation of HCV replication by AKT1 as compared to AKT2, suggesting that the inhibition of AKT1 specifically regulated HCV replication.

### DETAILED DESCRIPTION OF THE INVENTION

It is known that AKT phosphorylation motif is RXRXXS/T, which is present in AKT substrates. It is unexpectedly found in the present invention that as compared to those AKT substrates either of HCV 1b NS5B and NS3 has AKT-phosphorylation motif, RXRXXS/T, and various genotypes of HCV NS5B and NS3 have an AKT-phosphorylation motif, respectively.

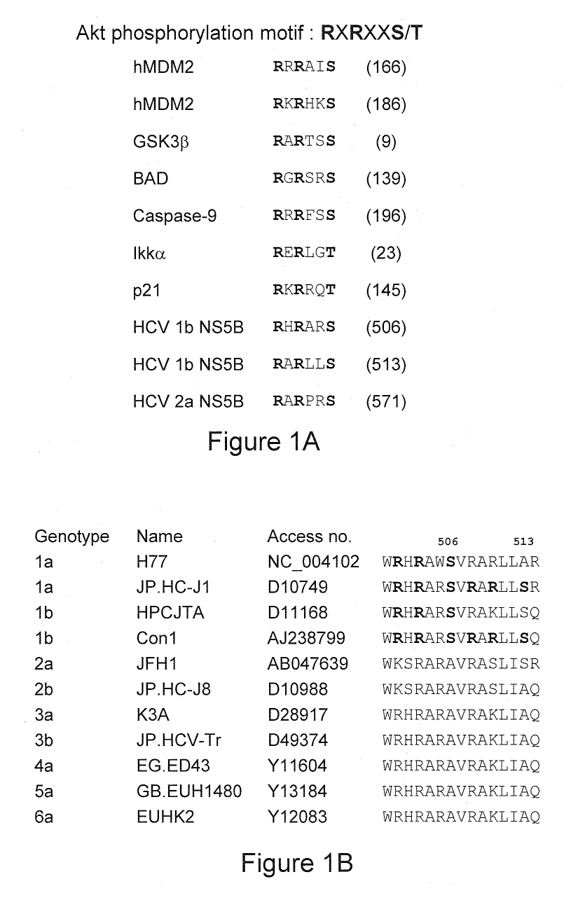

As shown in FIG. 1A, the amino-acid sequence of the AKT-phosphorylation motifs of HCV genotype 1b NS5B was compared to known AKT substrates. The consensus AKT phosphorylation motif was highlighted and shown at the top of FIG. 1A, wherein the numbers on the right indicated the positions of the final residues shown in each case. The alignment of putative AKT-phosphorylation motifs sequences among several genotypes of HCV NS5B was given FIG. 1B; wherein the numbers on the top indicated the positions of the potential phosphorylation residues shown in each case. The sequences as compared were obtained from HCV-1a (NC_{—}004102), HCV-1b (AJ238799), HCV-1c (D14853), HCV-2a (AB047645), HCV-2b (D10988), HCV-3a (D28917), HCV-3b (D49374), HCV-4a (Y11604), HCV-5a (Y13184), HCV-6b (009827).

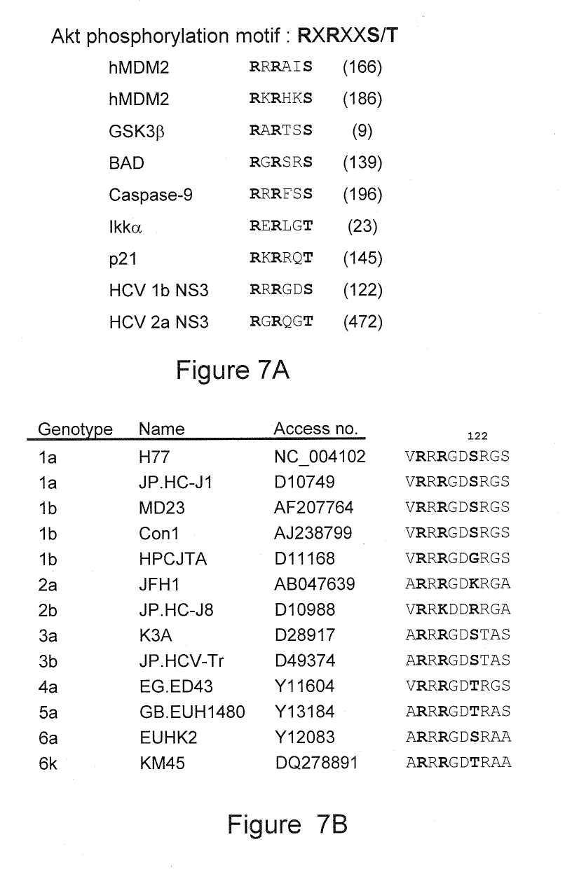

Similarly, as shown in FIG. 7A, HCV 1b NS3 has AKT-phosphorylation motif, RXRXXS/T, and several genotypes of HCV NS3S have an AKT-phosphorylation motif, evidenced by a comparison with other AKT substrates. An alignment of AKT-phosphorylation motifs sequences among several genotypes of HCV NS3 was given in FIG. 7B, wherein the numbers on the top indicated the positions of the potential phosphorylation residues shown in each case.

Given the finding above, it was hypothesized and evidenced in the present invention that AKT could enhance NS5B enzymatic activity in addition to that of NS3 through phosphorylating RXRXXS/T of these two HCV proteins. It was concluded in the present invention that to inhibit the activity of AKT or its activators could suppress NS3 and NS5B function and inhibit HCV genome replication, especially the genome type 1 and those with NS3 and NS5B processing this AKT phosphorylation motif. Therefore, inhibitors targeting AKT and its related activating pathways could serve as anti-HCV treatment in the presence or absence of simultaneous combination with inhibitors against NS5B and NS3.

As used herein, the term “activator of AKT” refers to any agent that provides the activity of AKT, including but not limited to Protein Kinase CK2, phosphoinositol-3-kinase, pyruvate dehydrogenase kinase, isozyme 1 etc.

Accordingly, the present invention provides a method for treating hepatitis C virus (HCV) infection. The method comprises administrating a subject in need thereof with an inhibitor of the activation of a serine/threonine kinase (AKT) and its activators, at an amount effective for blocking the function of NS3 or NS5B of HCV.

According to the invention, any AKT-specific inhibitor may serve a drug for treatment of HCV infection, including but not limited to the AKT-specific inhibitors listed by Kumar and Madison (Kumar and Madison, AKT crustal structure and AKT-specific inhibitors. Oncogene 24: 7493-7501, 2005), which is incorporated herein by reference in its entirety.

In another example of the invention, the inhibitor may be a small interfering RNA (siRNA) that could block the activation of AKT.

There are functional differences between AKT isoforms. It was observed that overexpression of AKT2, but not AKT1 is sufficient to restore insulin-mediated glucose uptake in AKT2^{-/-} adipocytes (Bae et al., Isoform-specific regulation of insulin-dependent glucose uptake by AKT/protein kinase B. J Niol Chem 278: 49530-49536, 2003). Therefore, an inhibitor of AKT2 activation should be avoided to minimize the side effect on insulin function.

According to the invention, the inhibition of AKT1 was compared with that of AKT2 in HCV replication, and it was unexpectedly found that AKT1 specifically regulated HCV replication, as compared with AKT2, as evidenced by the results of Example 10.

Accordingly, the invention provides a method for treating hepatitis C virus (HCV) infection with minimal side effects, comprising administrating a subject in need thereof with an agent that inhibits the activation of AKT1 but not AKT2, at an amount effective for blocking the function of NS3 or NS5B of HCV. In one example of the invention, the side effects on insulin function are minimized as avoiding any impact of AKT2 on insulin function.

According to the invention, the method for treating HCV infection may comprises co-administrating the subject with an anti-HCV drug. The anti-HCV drug may be any drug for treating HCV infection known or commonly used in the art.

On the other hand, the invention provides a method for screening a candidate agent for treating HCV infection, comprising:

determining the function of a candidate agent to inhibit the activity of a serine/threonine kinase (AKT) and detecting the presence or absence of an inhibition of the function of AKT; wherein the more function to inhibit the activation of AKT indicates the more activity in treating HCV infection.

Similarly, the invention provides a method for screening a candidate agent for treating HCV infection with minimal side effects, comprising: determining the function of a candidate agent to inhibit specifically the activity of AKT1 and AKT2; and detecting the presence or absence of an inhibition of the function of AKT1 and AKT2, respectively; wherein the more function to inhibit the activation of AKT1 but not AKT2 indicates the more activity in treating HCV infection with minimal side effects.

The present invention is further illustrated by the following examples, which are provided for the purpose of demonstration rather than limitation.

Materials and Methods

In Vivo Interaction of HCV NS5B/NS3 with AKT

Following transfection of AKT-HA for 48 hours, 293T cell lysates were precleared by incubation with 20 μl of 50% slurry of glutathione-agarose beads for 2 hours at 4° C. with end-over-end mixing. Approximately 1 μg of GST-NS5B-1bΔC21, GST-NS5B-2bΔC21, and GST control was incubated with 1 mg of precleaned 293T ell lysate followed by pull-down with GST resin. After washing with lysis buffer for 4 times, each bound protein was fractionated by SDS-PAGE and subjected to immunoblot with anti-HA antibody.

Recombinant Baculovirus Expressing HCV NS5B Protein

NS5B or NS5B (S506A) was subcloned using p3XFLAG-NS5B or p3XFLAG-NS5B (S506A) as a template into the XhoI and PstI sites of the pAcHLT-B transfer vector (BD) to obtain pAcHLT-B-FLAG-NS5B or pAcHLT-B-FLAG-NS5B (S506A). Recombinant baculovirus expressing HCV NS5B protein was produced according to manufacturer's manual with modification (BD). Briefly, 9×10⁵ Spodoptera frugiperda (Sf9) cells in 6-well culture plate were transfected with 0.15 μg of linearized BaculoGold DNA (Pharmingen, San Diego, Calif.), a modified Autographa californica nuclear polyhedrosis virus (AcNPV) DNA which contains a lethal deletion, together with 2 μg of either pAcHLT-B-FLAG-NS5B or pAcHLT-B-FLAG-NS5B (S506A). The transfected cells were incubated at 27° C. for 4 to 5 days and the supernatant was collected to infect more cells for amplification. The recombinant baculoviral NS5B proteins were purified. Protein concentrations were determined using a Bio-Rad protein assay kit with bovine serum albumin as a standard.

Immunoprecipitation and Immunoblotting

To determine whether NS5B interacts with AKT, 293T cells were co-transfected with 3 μg of pCMV-AKT-HA and 6 μg of p3XFLAG or p3XFLAG-NS5B plasmids and immunoprecipitation by anti-FLAG M2 resin or anti-HA followed by addition of protein A sepharose was performed followed by immunoblotting with anti-HA, anti-AKT or anti-FLAG antibody.

To map the interaction region of NS5B with AKT, full-length p3XFLAG-NS5B or its deletion mutants, p3XFLAG-NS5B (1-371) and p3XFLAG-NS5B (372-591), were co-transfected with pcDNA-AKT-HA into 293T cells. Immunoprecipitation with anti-FLAG M2 resin and immunoblotting with anti-HA antibody were performed.

Immunofluorescence

Huh7 cells plated in four-well chambered coverglass were co-transfected with pEGFP-NS5B or pEGFP vector and pcDNA3.1-AKT-HA plasmids for 48 hours. The cells were fixed in cold methanol and permeabilized in 0.2% Triton X100. Immunofluorescence staining was performed using anti-HA (1:200) as primary antibodies and rhodamine-conjugated donkey anti-rabbit IgG antibody (1:100; Jackson) as secondary antibodies. After washing, the cells were counterstained with 4′,6-Diamidino-2-phenylindole (DAPI) for nuclei staining. Confocal microscopy was performed using an Olympus IX 70 FLUOVIEW confocal microscope.

In Vitro Kinase Assay

In vitro kinase reaction was performed in 20 μl of kinase buffer containing 3 μg of purified NS5B or NS5B mutant (S506A) protein expressed by recombinant baculovirus, with or without 100 ng of activated AKT1 (Upstate Biotechnology), 200 μM ATP, and 10 μCi [γ-³²P]ATP (PerkinElmer Life Sciences) at 30° C. for 40 min. The reaction mixtures were stopped by sampling buffer and subjected to 10% SDS-PAGE. The phosphorylation of the fragments was detected by autoradiography.

RNA-Dependent RNA Polymerase (RdRP) Assay

RdRP activity of NS5B and NS5B mutant (S506A) was examined by the Poly(A)-dependent UMP Incorporation assay. One microgram of purified recombinant baculoviral NS5B was incubated at 22° C. for 2 h in the reaction solution (100 μl) containing 20 mM Tris-HCl (pH 7.5), 5 mm MgCl₂, 1 mm DTT, 25 mM KCl, 1 mM EDTA, 20 units of RNasin, 0.5 μCi of [³H]-UTP, 10 μm UTP, 10 μg/ml poly(A), and 200 nM oligo(U)₁₄. The RNA was precipitated by addition of 3 ml of 10% trichloroacetic acid (TCA) and incubated on ice for additional 15 min. The precipitate was filtered using GF-C filters followed by wash with 90% ethanol and air-dry. The filter-bound incorporated radiolabeled UTP was quantitated by scintillation counter.

Statistical Analysis

Statistical analysis was performed using the one-way ANOVA test as appropriate. p values less than 0.05 were considered as statistically significant.

### Example 1

### In Vivo Interaction of HCV NS5B with AKT

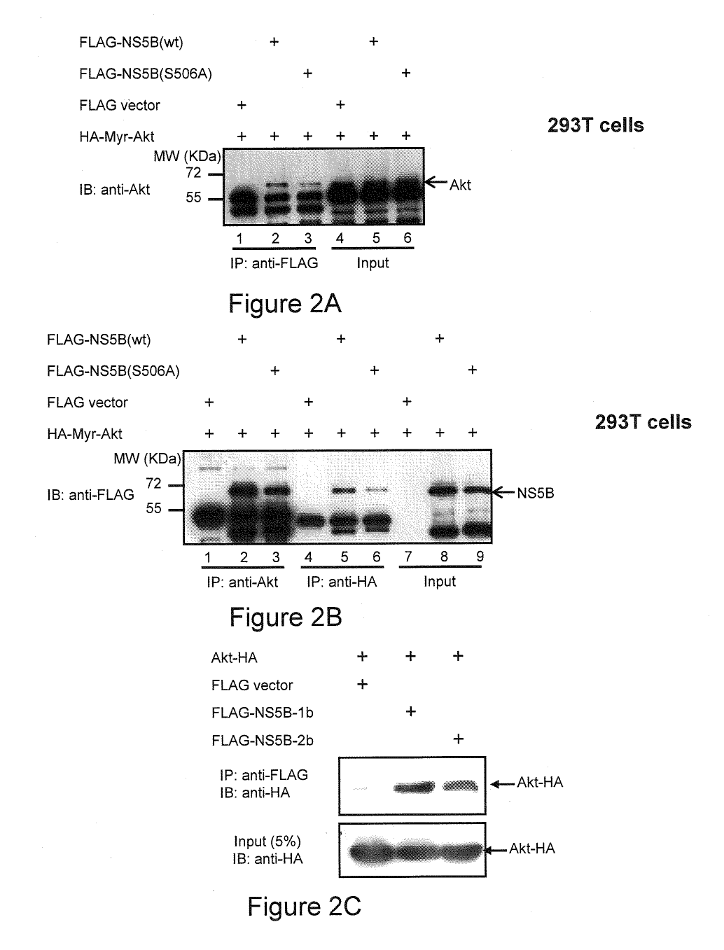

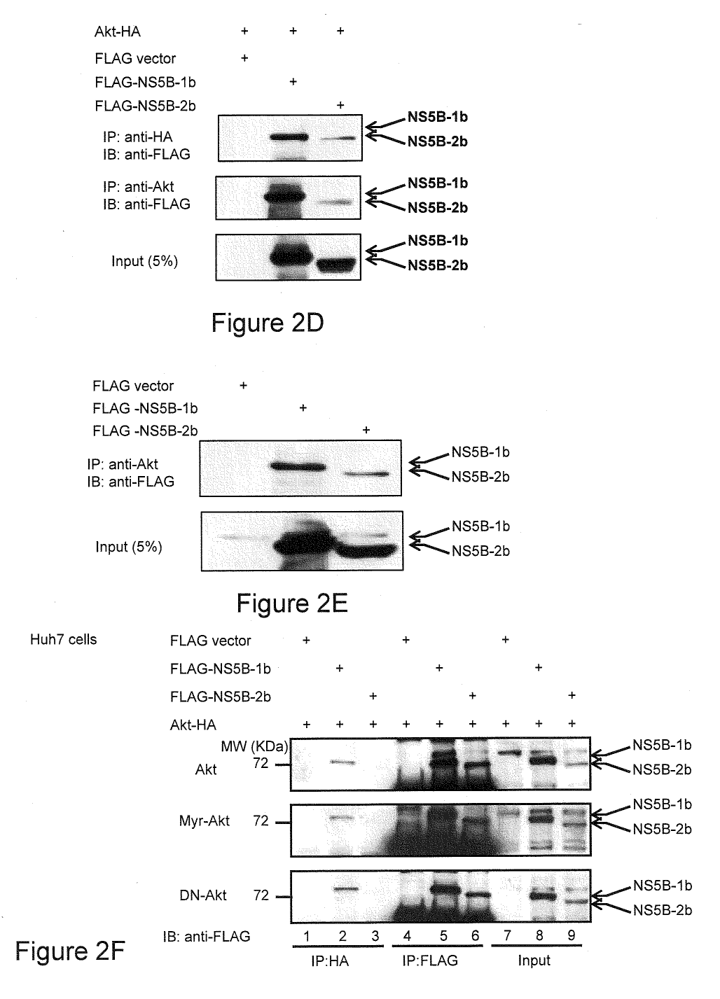

The association between HCV NS5B and AKT was shown on FIG. 2A, wherein Lanes 4, 5, and 6 represented 5% of the cell lysates used for coimmunoprecipitation.

On the other hand, the 293T cell lysates were immunoprecipitated with anti-AKT or anti-HA (3F10) antibody. After transfer to a nitrocellulose membrane, the bound NS5B was detected with FLAG-specific antibody. The results were shown in FIG. 2B, wherein Lanes 7, 8, and 9 represented 5% of the cell lysates used for coimmunoprecipitation. The flag-tagged NS5B-1b, NS5B-2b and FLAG vectors were co-transfected with HA-tagged AKT into Huh7 cells followed by immunoprecipitation of anti-FLAG resin and immunoblotting with anti-HA antibodies. The AKT was specifically immunoprecipitated by NS5B-1b and NS5B-2b but not vector control. The results were shown in FIG. 2C. The equal expression level of AKT representing 5% of the cell lysates used for coimmunoprecipitation was shown in lower panel. As shown in FIG. 2D, the Huh7 cell lysates following co-transfection of FLAG-NS5B-1b, FLAG-NS5B-2b or FLAG vector and AKT-HA were immunoprecipitated with anti-HA (upper panel) or anti-AKT (middle panel) antibodies. The bound NS5B-1b and, to a lesser extent, NS5B-2b but not vector control were detected by anti-FLAG antibody. The equal expression level of NS5B-1b and NS5B-2b representing 5% of the cell lysates used for coimmunoprecipitation was shown in lower panel in FIG. 2D.

The results of NS5B-1b, NS5B-2b and vector immunoprecipitated with anti-FLAG and immunoblotted with anti-HA antibodies were shown in FIG. 2E and the results of NS5B-1b, NS5B-2b and vector immunoprecipitated with AKT, Myr-AKT and DN-AKT and immunoblotted with anti-PLAG were given in FIG. 2F.

It is concluded that HCV NS5B would interact with AKT.

### Example 3

### In Vitro Interaction of HCV NS5B with AKT

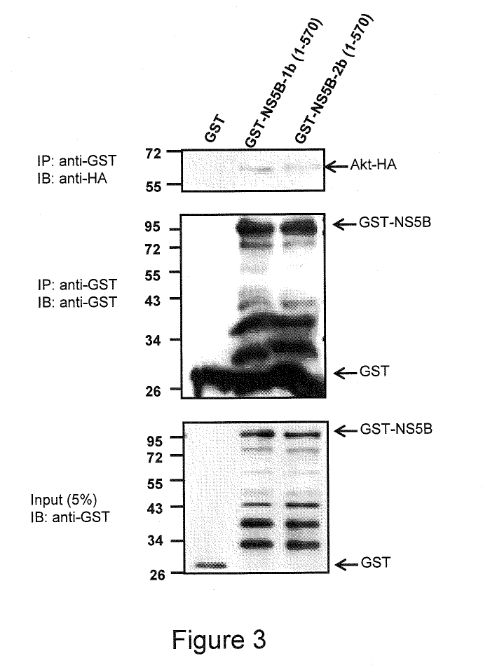

Following transaction of AKT-HA for 48 h, 293T cell lysates were precleared by incubation with 20 μl of a 50% slurry of glutathione-agarose beads for 2 h at 4° C. with end-over-end mixing. Approximately 1 μg of GST-NS5B-1bΔC21, GST-NS5B-2bΔC21, and GST control was incubated with 1 mg of precleaned 293T ell lysate followed by pull-down with GST resin. After washing with lysis buffer for 4 times, each bound protein was fractionated by SDS-PAGE, and were immunoblotted with anti-HA antibody (upper panel in FIG. 3). As shown in FIG. 3, the same membrane was re-probed with anti-GST antibody to show equal amount of GST or GST fusion protein was pulled down (middle panel), and 5% of the cell lysates used for GST pull-down assay (lower panel).

### Example 4

### Interaction of AKT with C-Terminus of HCV NS5B

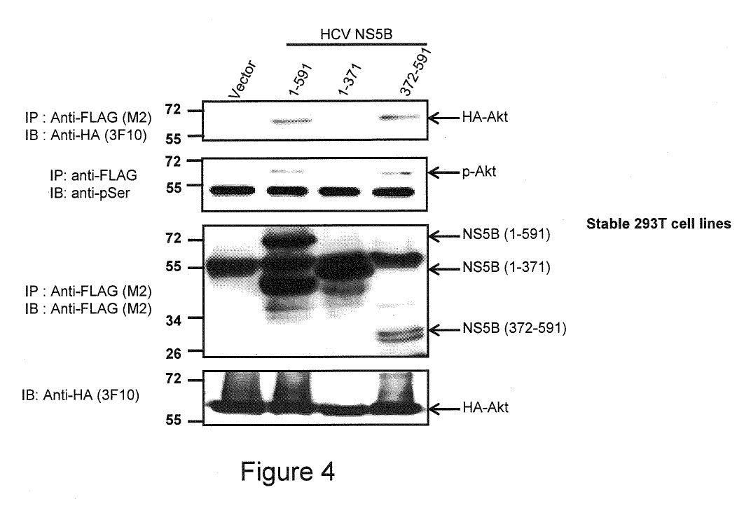

The pcDNA3.1-AKT-HA plasmid was transfected into stable 293T/FLAG, 293T/FLAG-NS5B (1-591), 293T/FLAG-NS5B (1-371), and 293T/FLAG-NS5B (372-591) cell lines followed by immunoprecipitation with anti-FLAG M2 resin and immunoblotting with anti-HA (3F10) and anti-phosphoserine (anti-pSer) antibodies. As shown in FIG. 4, specific co-immunoprecipitation of AKT-HA (upper panel) and phosphorylated AKT (second panel) were only shown by full-length (aa 1-591) and C-terminal of NS5B (aa 372-591) but not FLAG vector or N-terminal NS5B (1-371). The expression of full-length, N- and C-terminal of NS5B (third panel) and HA-AKT (lower panel) was also confirmed.

### Example 5

### Colorcalization of HCV NS5B Protein and AKT

The Huh7 cells were co-transfected with pEGFP-NS5B and pcDNA3.1-AKT-HA plasmids followed by staining with anti-HA as primary antibodies and rhodamine-conjugated donkey anti-rabbit IgG antibody as secondary antibodies. EGFP-NS5B and AKT-HA were visualized by confocal laser scanning microscopy, wherein green fluorescence indicated EGFP-NS5B protein, red fluorescence indicated AKT-HA, yellow fluorescence indicated EGFP-NS5B and AKT in the plasmamembrane regions. The 293T cells were co-transfected with pcDNA3.1-Myr-AKT-HA and pEGFP vector, pEGFP-NS5B (wt) or pEGFP-NS5B (S506A) plasmids. Two days after transfection, the cells were fixed. Immunofluorescence staining was performed using anti-HA as primary antibodies and rhodamine-conjugated donkey anti-rabbit IgG antibody as secondary antibodies. Confocal laser scanning microscopy revealed colorcalization of constitutively active AKT with NS5B (wt), and to a lesser extent, NS5B (S506A), in the plasmamembrane regions (right-hand side). Nuclei were visualized by DAPI staining.

### Example 6

### In Vitro Phosphorylation of NS5B at Serine 506 and Serine 513 by AKT

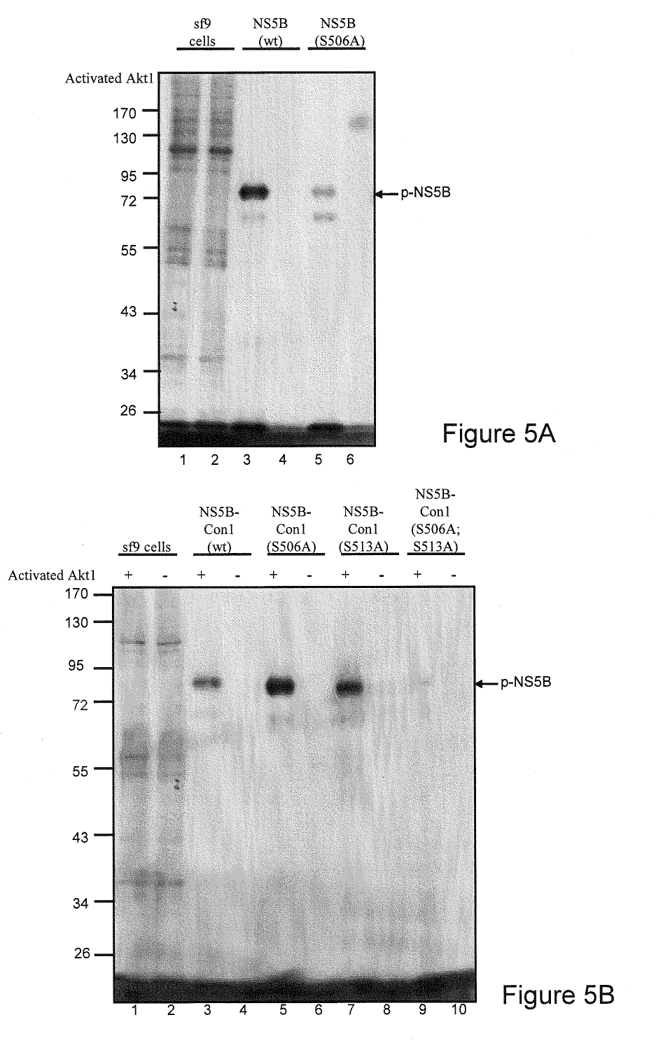

Three μg of purified recombinant either wild-type NS5B or mutant NS5B (S506A, S513A and S506,513AA) were incubated with or without 100 ng of activated AKT1 in a 20 kinase buffer containing 10 μCi [γ-³²P]ATP for 40 min at 30° C. The kinase reaction was stopped by the addition of SDS-PAGE sampling buffer and subjected to SDS-PAGE (10% gel), and analyzed by autoradiography. As shown in FIG. 5, the arrows indicated the positions of NS5B phosphorylated by AKT and autophosphorylated AKT. Phosphorylation of wild-type NS5B, NS5B (S506A), and NS5B (S513A) by AKT was clearly observed (Lane 3, 5 and 7) while NS5B carrying the double mutations at the potential AKT phosphorylation consensus motif S506,513AA) entirely prevented AKT phosphorylation (Lane 9), and Sf9 cell lysates were used as negative control (Lane 1 and 2).

### Example 7

### In Vivo Phosphorylation of NS5B In Vivo

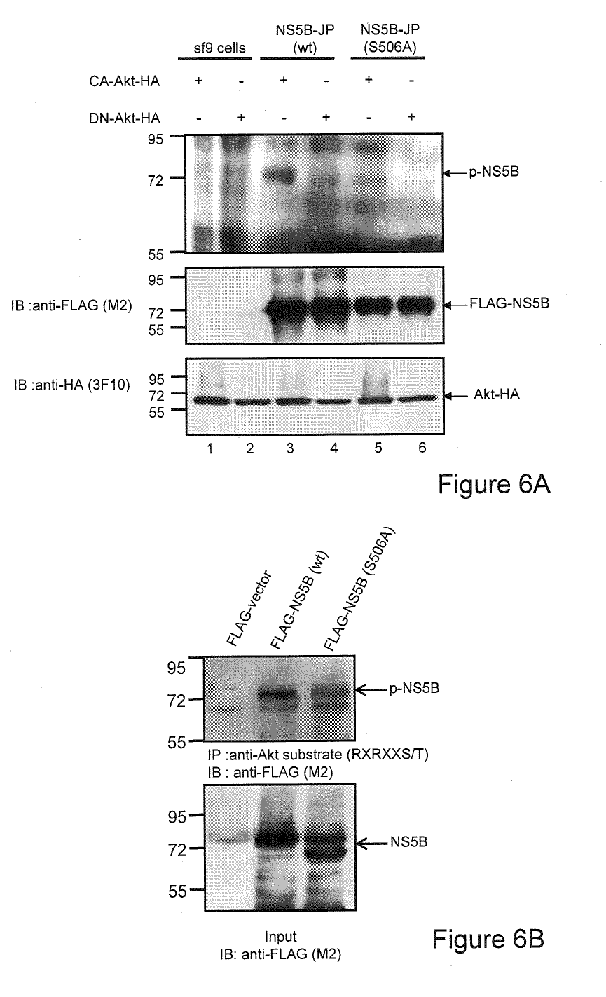

Stable 293T/FLAG and 293T/FLAG-NS5B were lysed and followed by immunoprecipitation with anti-FLAG M2 resin and immunoblotting with anti-phosphoserine (pSer-45) antibodies. As shown in FIG. 6A, phosphorylated NS5B was specifically immunoprecipitated from 293T/FLAG-NS5B cell lysates (upper panel), the membrane was reprobed with anti-FLAG M2 antibodies to show NS5B expression in 293T/FLAG-NS5B cells (lower panel). Molecular mass standards are shown on the left in FIG. 6B. The stable 293T/FLAG and 293T/FLAG-NS5B were lysed and followed by immunoprecipitation with anti-FLAG M2 resin and immunoblotting with Phospho-AKT Substrate (RXRXXS/T) Rabbit mAb antibodies, and phosphorylated NS5B was specifically immunoprecipitated from 293T/FLAG-NS5B cell lysates (upper panel). The membrane was reprobed with anti-FLAG M2 antibodies to show NS5B expression in 293T/FLAG-NS5B cells (middle panel), the equal expression of phosphorylated AKT was shown in immunoblot of input using anti-phospho-AKT (S473) antibodies (lower panel).

### Example 8

### In Vivo Interaction of HCV NS3 M-Terminal Region with AKT

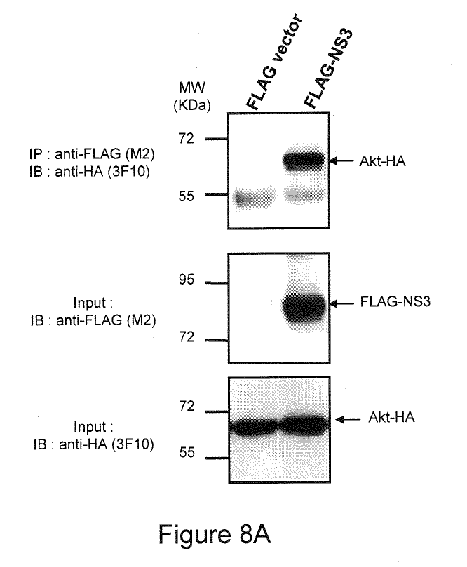

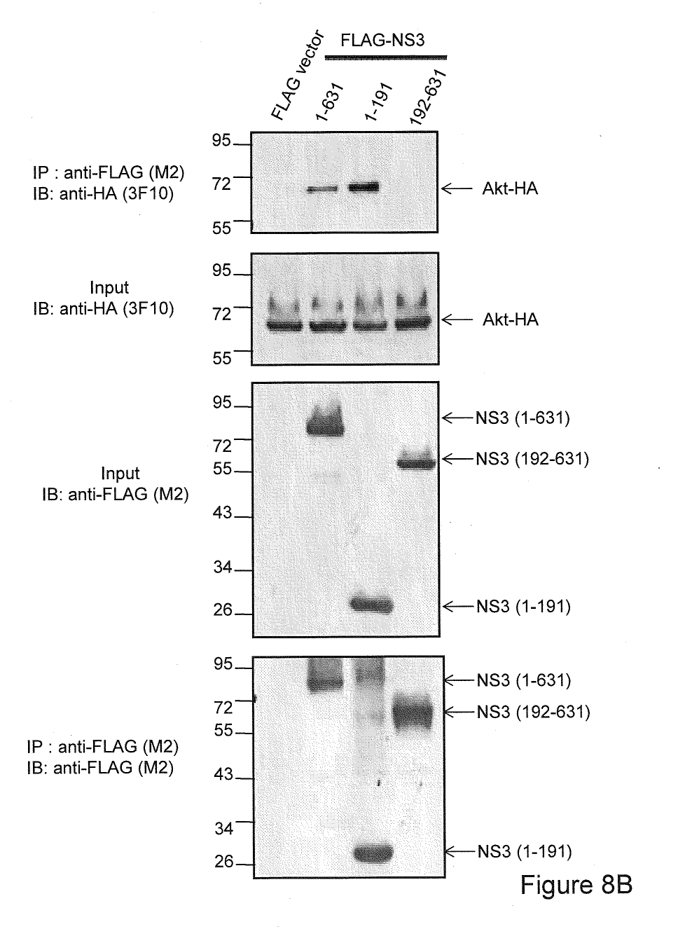

Flag-tagged HCV NS3 or vector and HA-tagged AKT were co-transfected into 293T cells. The cells were lysed 48 h later and NS3 was immunoprecipitated (IP) with anti-FLAG-M2 (Sigma) antibody. After transfer to a nitrocellulose membrane, AKT was immunoblotted (IB) with anti-HA (3F10) antibody. The results were given in FIG. 8A. To determine the region required for NS3 interaction with AKT, similar immunoprecipitation experiment were conducted as in FIG. 8A using vectors of flag-tagged constructs encoding various regions of NS3 with full length (1-631, 1-191, 192-631) cotransfected with HA-tagged AKT into 293T cells. The 293T cell lysates were lysed 48 h after transfection and NS3 was immunoprecipitated (IP) with anti-FLAG-M2 (Sigma) antibody. After transfer to a nitrocellulose membrane, AKT was immunoblotted (IB) with anti-HA (3F10) antibody as shown in FIG. 8B.

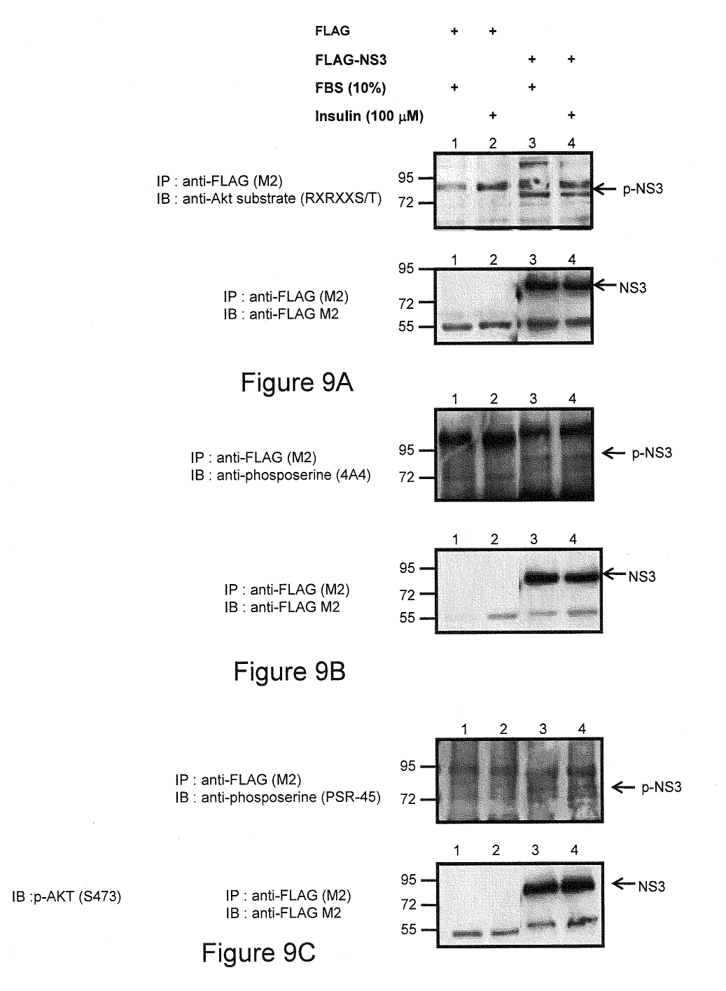

The phosphorylation in AKT phosphorylation motif RXRXXS/T was performed to determine where NS3 was phosphorylated. It was found that the NS3 was phosphorylated at serine residue within the RXRXXS/T AKT phosphorylation motif (see FIG. 9).

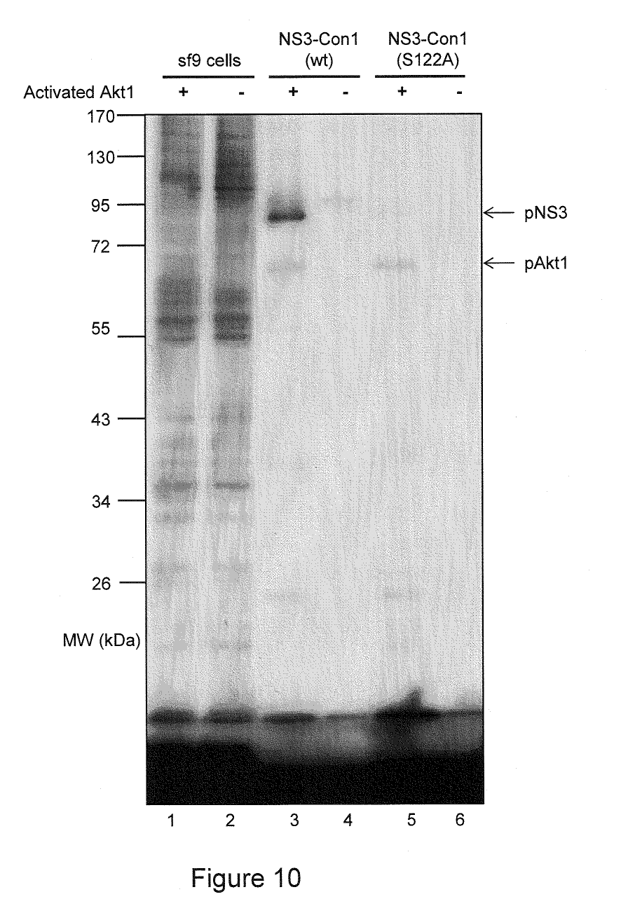

Furthermore, the phosphorylation of Serine 122 in the AKT-phosphorylation motif RXRXXS/T of NS3 by active AKT was confirmed. Three μg of purified recombinant either wild-type NS3 or mutant S122A NS3 were incubated with or without 100 ng of activated AKT1 in a 20 μl kinase buffer containing 10 μCi [γ-³²P]ATP for 40 min at 30° C. The kinase reaction was stopped by the addition of SDS-PAGE sampling buffer and subjected to SDS-PAGE (10% gel), and analyzed by autoradiography. As shown in FIG. 10, the arrows indicated the positions of NS3 phosphorylated by AKT and autophosphorylated AKT. As shown in FIG. 10, the phosphorylation of wild-type NS3 by AKT was clearly observed in the presence of active AKT (Lane 3) while mutation of NS3 Serine 122 to Alanine in the AKT phosphorylation consensus motif entirely prevented AKT phosphorylation (Lane 5 and 6), and Sf9 cell lysates were used as negative control (Lane 1 and 2).

### Example 9

### NS3 Activity was Suppressed by Elimination of AKT-Phosphorylation Motif

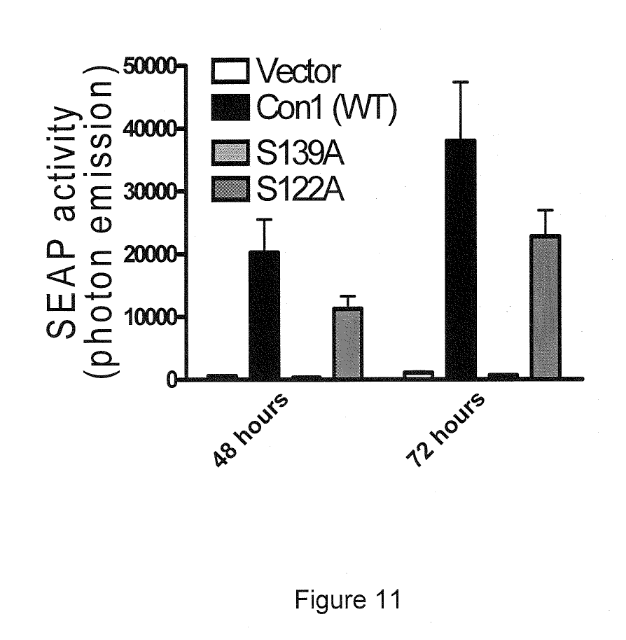

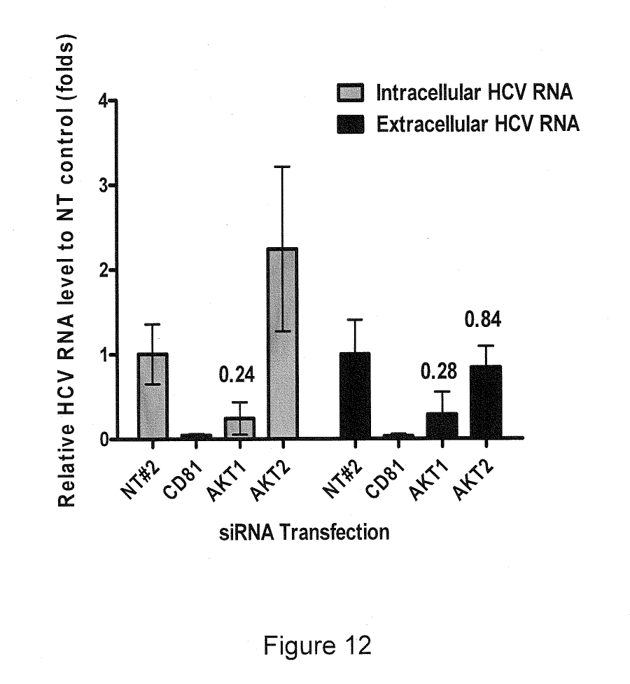

To examine the activity of NS3 in cell culture, a substrate vector, pEG(D4AB)SEAP, encoding enhanced green fluorescent protein (EGFP) and secreted alkaline phosphatase (SEAP) chimera with NS3/4A protease decapeptide recognition sequence linker in-betweens (20) was co-transfected into 293T cells with wild type (WT), S139A, or S122A NS3 in p3XFlag expression vector. The amount and activity of secreted SEAP reflects the activity of various NS3 proteins. Vector and enzyme-dead S139A mutant constructs only showed background SEAP activity 48 and 72 hours after transfection. As shown in FIG. 11, AKT-phosphorylation motif eliminated S122A mutant demonstrated suppressed SEAP activity as compared with wild type, indicating decreased NS3 activity in the absence of AKT phosphorylation of S122.

### Example 10

### Suppression of HCV Replication by the Inhibition of AKT1 and AKT2

The purpose of this example was to compare the inhibition effects of AKT1 and AKT2 in HCV replication. Accordingly, siRNAs were transfected into the Huh 7.5.1 cells at a 50 nM final concentration, using Oligofectamine (Invitrogen) in a 24-well format. After 72 h of siRNA-mediated gene knockdown, the medium was removed and the cells were infected with HCV and incubated overnight. Then, the media was replaced with fresh medium, and after an additional 48-hour incubation, the supernatant was collected for extraction of extracellular HCV RNA using QIAamp Viral RNA Mini Kit (QIAGEN, USA) and the cells were harvested for extraction of intracellular HCV RNA using RNeasy mini kit (QIAGEN, USA). The copy numbers of HCV RNA were determined by quantitative PCR using the TaqMan EZ RT-PCR CORE REAGENTS (Applied Biosystems, USA) on an ABI 7500 Real Time PCR System (Applied Biosystems, USA).

The siRNAs against CD81 which resulted in inhibition of intracellular, in turn, extracellular HCV RNA, was served as positive control. The siRNAs against AKT1, but not AKT2, also inhibited HCV propagation as reflected by suppression of both intracellular and extracellular HCV RNA by 4 fold as compared to non-targeting (NT#2) control. The results were shown in Figure, suggesting that AKT1 played a specific role in infectious HCV particle formation. It was concluded that AKT1 specifically regulated HCV replication, as compared with AKT2.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A method for treating hepatitis C virus (HCV) infection, comprising administrating a subject in need thereof with an inhibitor of the activation of a serine/threonine kinase (AKT) and its activators, at an amount effective for blocking the function of NS3 or NS5B of HCV.

2. The method of claim 1, wherein the inhibitor is a small interfering RNA (siRNA) that could block the activation of AKT.

3. A method for treating hepatitis C virus (HCV) infection with minimal side effects, comprising administrating a subject in need thereof with an agent that inhibits the activation of AKT1 but not AKT2, at an amount effective for blocking the function of NS3 or NS5B of HCV.

4. The method of claim 3, wherein the agent is a small interfering RNA (siRNA) that could block the activation of AKT.

5. A method for screening a candidate agent for treating hepatitis C virus (HCV) infection, comprising:
determining the function of a candidate agent to inhibit the activity of a serine/threonine kinase (AKT) and detecting the presence or absence of an inhibition of the function of AKT; wherein the more function to inhibit the activation of AKT indicates the more activity in treating HCV infection.

6. A method for screening a candidate agent for treating hepatitis C virus (HCV) infection with minimal side effects, comprising: determining the function of a candidate agent to inhibit specifically the activity of AKT1 and AKT2; and detecting the presence or absence of an inhibition of the function of AKT1 and AKT2, respectively; wherein the more function to inhibit the activation of AKT1 but not AKT2 indicates the more activity in treating HCV infection with minimal side effects.

7. The method of claim 1, wherein the AKT activator is one selected from the group consisting of Protein Kinase CK2, phosphoinositol-3-kinase, pyruvate dehydrogenase kinase, isozyme 1, ErbB-3, ataxia telangiectasia, ATM, Proline-rich tyrosine kinase 2 (Pyk2), mTOR, and receptors activating phosphoinositol-3-kinase, intergrin and combination thereof.

8. The method of claim 7, wherein the receptors activating phosphoinositol-3-kinase is G-protein-coupled receptor, a growth factor receptor, or a cytokine receptor.
